(19) United States
(12) Patent Application Publication
(10) Pub. No.: US 2007/0282181 A1
(43) Pub. Date: Dec. 6, 2007

(54) Visual medical sensor indicator

(76) Inventors: Carol Findlay, San Ramon, CA (US); William L. Shea, Los Gatos, CA (US)

Correspondence Address: Nellcor Puritan Bennett LLC;c/o Fletcher Yoder PC, P.O. BOX 692289, HOUSTON, TX, 77269-2289, US

(21) Appl. No.: 11/444,725

(22) Filed: Jun. 1, 2006

(51) Int. Cl. A61B 5/00

(52) U.S. Cl. 600/323

(57) ABSTRACT

The disclosure includes a medical device having a disposable, replaceable or reusable medical sensor and a visual indicator of at least one condition. The disclosure also includes a method of determining exposure of a medical sensor to a condition by viewing a visual indicator of the condition on a medical sensor and, based upon viewing of the visual indicator, determining whether the medical sensor has been exposed to the condition.

### TECHNICAL FIELD

The present disclosure, according to one embodiment, relates to a visual medical sensor indicator to provide information regarding medical sensor replacement or disposal.

### BACKGROUND

This section is intended to introduce the reader to various aspects of art that may be related to various aspects of the present invention, which are described and/or claimed below. This discussion is believed to be helpful in providing the reader with background information to facilitate a better understanding of the various aspects of the present invention. Accordingly, it should be understood that these statements are to be read in this light, and not as admissions of prior art.

Sensors are increasingly common in medical monitoring and diagnostics. The combination of advances in electronics, chemistry and our understanding of diseases and physiological conditions has made possible far more sophisticated observations of patient condition through the development of a wide array of medical sensors. Some sensors interface with electronic components that use computer technology to perform complex data analysis. Others use advances in chemicals to provide simple visual cues indicating the patient's condition. Some of these sensors are self-contained units, while others involve various parts or may be combined with other medical devices. However, most sensors involve at least one component designed to be replaced after one use, between patients, after a certain time period, or when other conditions are met.

### SUMMARY

Certain aspects commensurate in scope with the originally claimed invention are set forth below. It should be understood that these aspects are presented merely to provide the reader with a brief summary of certain forms the invention might take and that these aspects are not intended to limit the scope of the invention. Indeed, the invention may encompass a variety of aspects that may not be set forth below.

According to one embodiment, the disclosure relates to a medical device having a disposable, replaceable or reusable medical sensor and a visual indicator of at least one condition of the sensor.

According to another embodiment, the disclosure relates to a medical device having a disposable, replaceable, or reusable medical sensor and a visual indicator means operable to indicate at least one condition.

According to a third embodiment the disclosure relates to a method of determining exposure of a medical sensor to a condition by viewing a visual indicator of the condition on a medical sensor, and based upon viewing the visual indicator, determining whether the medical sensor has been exposed to the condition.

### BRIEF DESCRIPTION OF THE DRAWINGS

A more complete understanding of the present disclosure may be acquired by referring to the following description taken in conjunction with the accompanying drawings wherein:

FIG. 1 is a schematic diagram of a medical sensor with a visual indicator, according to a specific example embodiment of the present disclosure; and

FIG. 2 is a flow chart of a method of triggering a visual indicator on a medical sensor, according to a specific example embodiment of the present disclosure; and

FIG. 3 is a flow chart of another method of triggering a visual indicator on a medical sensor, according to a specific example embodiment of the present disclosure.

While the invention is susceptible to various modifications and alternative forms, specific embodiments have been shown by way of example in the drawings and will be described in detail herein. However, it should be understood that the invention is not intended to be limited to the particular forms enclosed. Rather, the invention is to cover all modifications, equivalents and alternatives falling within the spirit and scope of the invention as defined by the appended claims.

### DETAILED DESCRIPTION

Referring now to the drawings, the details of specific example embodiments are schematically illustrated. Like elements in the drawings will be represented by like numbers, and similar elements will be represented by like numbers with a different lower case letter suffix.

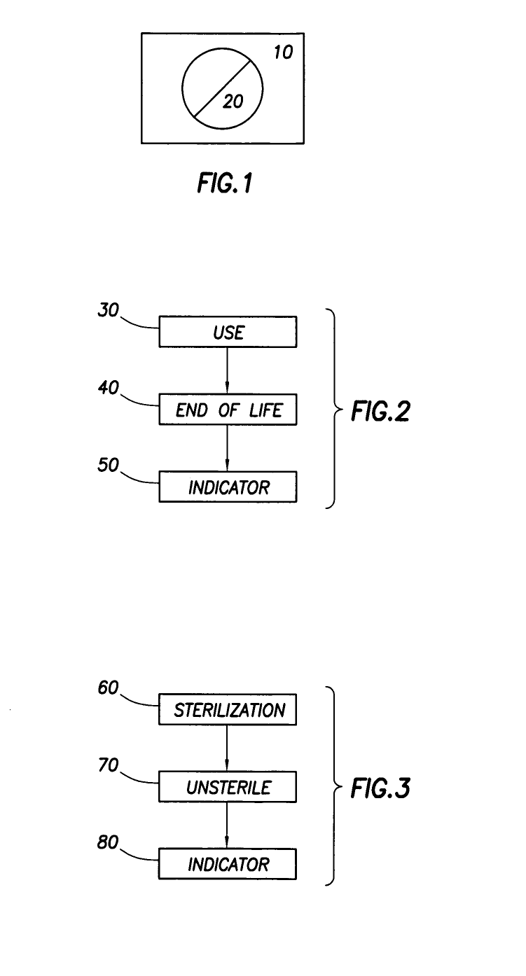

Referring to FIG. 1, depicted is a schematic block diagram of medical sensor 10 with visual indicator 20. Medical sensor 10 may be any type of medical sensor whose disposal or replacement may be advisable. In particular, it may be a disposable, replaceable or reusable medical sensor. The sensor may be a self-contained unit, or a component of a medical device. The sensor may contain non-sensing elements, e.g., a bandage, adhesive and/or a housing. If the sensor is reusable, it may be designed for particular treatment, such as sterilization, before reuse.

Medical sensor 10 may include or be a component of an oximetry sensor (e.g., a pulse oximetry sensor), air and/or breath sensors (e.g., a carbon dioxide sensor), a temperature sensor, a pH sensor, a spirometer, a flow sensor, a catheter (e.g., an intrauterine pressure catheter, an umbilical vessel catheter, and/or a replogle suction catheter), a nasal cannula, an electrode (e.g., an ECG electrode, and/or a fetal spinal electrode system), a saturation BMP sensor, needles (e.g., biopsy needles), airway management products (e.g. laryngeal masks and adaptors, AMBU bags with masks and intubation tubes), blood pressure cuffs, or tissular water sensors, by way of example.

Visual indicator 20 may be formed from any material compatible with medical sensor 10 and operable to provide desired detection and/or indication functions. Visual indicator 20 may include multiple indicators which may be in different, the same, or overlapping locations on medical sensor 10. Specifically, visual indicator 20 may be a chemical (e.g., a chemical sensitive to oxygen, another gas or air, a chemical that decays after a given time period, a chemical sensitive to light, a chemical sensitive to humidity, a chemical sensitive to temperature, a chemical sensitive to the presence of another chemical, and/or a chemical sensitive to a pathogen) and/or a mechanical component (e.g., part of a bandage and/or a flex indicator).

Visual indicator 20 may be any suitable natural or man-made material. In particular embodiments in which visual indicator 20 may be a mechanical component, it may include a creasable material, for example a polymer, a plastic, a shape-memory compound, and/or a thermoplastic material. In particular embodiments in which visual indicator 20 may be a chemical, it may include dyes, for example chromogenic dyes, pigments, fluorescent chemicals, phosphorescent chemicals, thermosensitive chemicals, electro-sensitive chemicals reactive chemicals, pathogen-sensitive chemicals, and/or pH-sensitive chemicals. Chemical indicators may include more than one distinct chemical species and may include additional chemicals, such as buffers, that do not have a visible color.

Visual indicator 20 may form an integral part of medical sensor 10, or it may be attached to medical sensor 10. For example, it may be affixed, e.g., using an adhesive, sonic welding and/or by melting, Velcro®, or it may be associated with medical sensor 10 through a tether, e.g., a plastic cord or string, through a snap or button, slipped into a carrier/pocket, or by sewing.

Visual indicator 20 may be operable to detect a variety of conditions. These conditions may include exposure to oxygen, another gas or air, compromise of sterility, e.g., rupture of packaging after sterilization, failure of adhesive, mechanical fatigue and/or wear, passage of time, exposure to temperature above, below or outside of an acceptable range, exposure to humidity above, below or outside of an acceptable range, exposure to light, e.g., exposure to light of a particular intensity and/or for a particular duration, exposure to inappropriate chemicals, e.g., chemical indicative of attempts to clean a disposable device, exposure to blood, food, urine and or feces, exposure to a pathogen, exposure to radiation, and/or other conditions that affect sensor function.

Visual indicator 20 may, in some embodiments, become or remain substantially visible when conditions negatively affecting sensor function occur. Alternatively, visual indicator 20 may, in other embodiments, become or remain substantially visible when conditions positively affecting sensor function occur. Additionally, in some embodiments, visual indicator 20 may be visible under both positive and negative conditions, but undergo a detectable visual change when conditions change. In some embodiments, “visible” may refer to readily visible to a human with normal vision, and may exclude indicator states that may only be detectable through close visual scrutiny or through use of special “detection” equipment/device.

Visual indicator 20 may provide visual indication through appearance or disappearance of a marking, change in number of markings, or change in color. It may also glow in the dark, which may assist in monitoring of the indicator without disturbing patients, particularly patients in darkened rooms. It may be in any form, including the “do not use” symbol shown in FIG. 1, creases in a bandage or component added to a bandage which may show the number of times the bandage has been moved, or tick marks, which may indicate, after a certain number have appeared or disappeared, that disposal or replacement of medical sensor 10 is recommended. Visual indicator 20 may also deform otherwise change shape, e.g. in when a current is passed through an electro-sensitive chemical. Visual indicator 20 may be selected to allow quick inspection and/or to provide confidence that medical sensor 10's capacity has not been compromised. Such functions may be particularly useful in emergency medical diagnostics and/or monitoring. Visual indicator 20 may also be selected to provide information useful to technical support and service personnel.

Disposal or replacement of medical sensor 10 may be desirable as a result of a variety of conditions, including use once or a set number of times, passage of time, depletion of a limited component, and/or exposure to potentially damaging and/or non-sterile conditions. For example, in the method shown in FIG. 2, medical sensor 10 is used in step 30, at some time point in step 40 medical device 10 reaches the end of its usable life. This may be due, for example, to passage of time, single use, depletion of a limited component, and/or exposure to adverse conditions during use. Shortly after step 40, visual indicator 50 indicates in step 50 that medical sensor 10 has reached the end of its life and should be replaced or disposed of.

Similarly, in the method shown in FIG. 2, medical sensor 10 is sterilized in step 60, at some later time, medical sensor 10 becomes non-sterile in step 70, shortly thereafter, in step 80, visual indicator 20 indicates this change in sterility condition, which may indicate that recleaning or disposal of medical device 10 is recommended. Visual indicator 20 may also be resettable, e.g. when medical sensor 10 is resterilized. In the case of resettable sensors, the visual sensor 20 may indicate that actions to reset the sensor may be recommended.

In more particular embodiments, medical sensor 10 may be a carbon dioxide sensor and visual indicator 20 may detect exposure of the sensor to excess humidity, which may negatively affect its ability to accurately detect carbon dioxide. Visual indicator 20 may optionally also indicate when the product has reached a certain age, which may also negatively affect its ability e.g., to accurately detect carbon dioxide.

In other embodiments, medical sensor 10 may be an endotracheal tube and visual indicator 20 may detect compromise of sterility, for example by exposure to oxygen, another gas, air, and/or chemicals. Visual indicator 20 may also detect exposure to other sterility compromising agents, such as blood, food, feces, urine and/or pathogens. In some embodiments, visual indicator 20 may be primarily functional to detect the presence or exposure to particularly dangerous agents, such as blood and/or pathogens, rather than general compromise of sterility.

In yet another embodiment, visual indicator 20 may indicate that medical sensor 10 should be repositioned or replaced due to passage of a certain amount of time. This function may be particularly useful in sensors used for patients with sensitive skin, for example, some elderly, infants, and burn patients and other patients with skin conditions e.g. a rash or skin disease.

In another embodiment, visual indicator 20 might help a user determine the age of medical sensor 10 prior to undertaking time consuming or costly preparation of that sensor, such as sterilization. For example, visual indicator 20 may indicate if medical sensor 10 has reached a certain age.

In another embodiment, visual indicator 20 may indicate that medical sensor 10 is applied with too much or too little force, thus indicating the misuse/misapplied application of the sensor to device. For example, visual indicator 20 may be pressure-sensitive and located in a region of medical sensor 10 where application force is also applied to visual indicator 20.

While embodiments of this disclosure have been depicted, described, and are defined by reference to specific example embodiments of the disclosure, such references do not imply a limitation on the disclosure, and no such limitation is to be inferred. The subject matter disclosed is capable of considerable modification, alteration, and equivalents in form and function, as will occur to those ordinarily skilled in the pertinent art and having the benefit of this disclosure. For example, many different combinations of sensors with visual indicators of different colors and forms and sensitive to various conditions may be envisioned by one skilled in the art and are intended to be included within the scope of this disclosure. It should be understood that various changes, substitutions and alterations can be made herein without departing from the spirit and scope of the disclosure as illustrated by the following claims.

What is claimed is:

1. A medical device comprising:
a disposable, replaceable, or reusable medical sensor; and
a visual indicator of at least one condition of the sensor.

2. The medical device according to claim 1, wherein the medical sensor comprises a self-contained unit.

3. The medical device according to claim 1, wherein the sensor comprises an oximetry sensor.

4. The medical device according to claim 1, wherein the sensor comprises an air or breath sensor.

5. The medical device according to claim 1, wherein the sensor comprises a temperature sensor.

6. The medical device according to claim 1, wherein the sensor comprises a pH sensor.

7. The medical device according to claim 1, wherein the sensor comprises a catheter.

8. The medical device according to claim 1, wherein the sensor comprises an electrode.

9. The medical device according to claim 1, wherein the sensor comprises a needle.

10. The medical device according to claim 1, wherein the sensor comprises a tissular water sensor.

11. The medical device according to claim 1, wherein the sensor comprises a spirometer.

12. The medical device according to claim 1, wherein the sensor comprises a flow sensor.

13. The medical device according to claim 1, wherein the sensor comprises a saturation BMP sensor.

14. The medical device according to claim 1, wherein the visual indicator comprises multiple indicators.

15. The medical device according to claim 1, wherein the visual indicator comprises a chemical.

16. The medical device according to claim 15, wherein the condition comprises at least one of oxygen, air, a gas other than oxygen or air, passage of time, light, humidity, temperature, or presence of another chemical or a pathogen.

17. The medical device according to claim 15, wherein the chemical comprises at least one of dyes, chromogenic dyes, pigments, fluorescent chemicals, phosphorescent chemicals, thermosensitive chemicals, pH-sensitive chemicals, reactive chemicals, or pathogen-sensitive chemicals.

18. The medical device according to claim 1, wherein the visual indicator comprises multiple distinct chemical species.

19. The medical device according to claim 1, wherein the visual indicator comprises a mechanical component.

20. The medical device according to claim 19, wherein the mechanical component comprises part of a bandage or a flex indicator.

21. The medical device according to claim 19, wherein the mechanical component comprises at least one of creasable materials, polymers, plastics, shape-memory compounds, or thermoplastic materials.

22. The medical device according to claim 1, wherein the visual indicator forms an integral part of the medical sensor.

23. The medical device according to claim 1, wherein the visual indicator is affixed to or associated with to the medical sensor.

24. The medical device according to claim 1, wherein the condition comprises at least one of exposure to oxygen, air, or a gas other than oxygen or air, compromise of sterility, rupture of packaging after sterilization, failure of adhesive, mechanical fatigue or wear, passage of time, exposure to temperature above, below or outside of an acceptable range, exposure to humidity above, below or outside of an acceptable range, exposure to light, exposure to light of a particular intensity or for a particular duration, exposure to inappropriate chemicals, or exposure to blood, food, urine, feces or a pathogen,.

25. A medical device comprising:
means for medically sensing a parameter of a patient; and
means for visually indicating at least one condition of the sensing means.

26. The medical device according to claim 25, wherein the condition comprises at least one of exposure to oxygen, air, or a gas other than oxygen or air, compromise of sterility, rupture of packaging after sterilization, failure of adhesive, mechanical fatigue or wear, passage of time, exposure to temperature above, below or outside of an acceptable range, exposure to humidity above, below or outside of an acceptable range, exposure to light, exposure to light of a particular intensity or for a particular duration, exposure to inappropriate chemicals, or exposure to blood, food, urine, feces, or a pathogen.

27. A method of determining exposure of a medical sensor to a condition comprising:
viewing a visual indicator of the condition on a disposable, replaceable, or reusable medical sensor; and
based upon viewing of the visual indicator, determining whether the medical sensor has been exposed to the condition.

28. The method according to claim 27, wherein the visual indicator becomes visible upon occurrence of the condition.

29. The method according to claim 27, wherein the visual indicator becomes invisible upon occurrence of the condition.

30. The method according to claim 27, wherein the visual indicator changes color upon occurrence of the condition.

31. The method according to claim 27, wherein the visual indicator changes shape upon occurrence of the condition.

32. The method according to claim 27, wherein the condition negatively affects the function of the medical sensor, the method comprising disposing of the medical sensor if the visual indicator indicates that the condition has occurred.

33. The method according to claim 27, wherein the condition positively affects the function of the medical sensor, the method comprising disposing of the medical sensor unless the visual indicator indicates that the condition has occurred.

34. The method according to claim 27, wherein the condition is comprises at least one of exposure to oxygen, air, or a gas other than oxygen or air, compromise of sterility, rupture of packaging after sterilization, failure of adhesive, mechanical fatigue or wear, passage of time, exposure to temperature above, below or outside of an acceptable range, exposure to humidity above, below or outside of an acceptable range, exposure to light, exposure to light of a particular intensity or for a particular duration, exposure to inappropriate chemicals, or exposure to blood, food, urine, feces, or a pathogen.
